(19) United States
(12) Patent Application Publication
(10) Pub. No.: US 2023/0043560 A1
(43) Pub. Date: Feb. 9, 2023

(54) CLEANABLE RESPIRATORY CIRCUIT

(71) Applicant: Owen Giesbrecht, Coaldale (CA)

(72) Inventor: Owen Giesbrecht, Coaldale (CA)

(21) Appl. No.: 17/791,719

(22) PCT Filed: Jan. 8, 2020
(86) PCT No.: PCT/CA2020/050019

§ 371 Date: Jul. 8, 2022

(51) Int. Cl. A61M 16/08

(52) U.S. Cl. CPC A61M 16/0875

(57) ABSTRACT

A cleanable respiratory tube is provided. The cleanable respiratory tube comprises a corrugated tube adapted for conveying gases to and/or from a patient. The corrugated tube has a first and a second fitting at a first and a second end thereof, respectively. A sheath having a smooth cleanable outer surface encases the corrugated tube. A first and a second end of the sheath is fixedly attached to the respective first and second fitting. The sheath is made of a flexible foil-type sheet material surrounding the corrugated tube having an inside diameter approximately equal to an outside diameter of ridges of the corrugated tube such that the sheath is in a touching relationship therewith.

### FIELD OF THE INVENTION

The present invention relates to respiratory tubes for conveying gases to and/or from a patient, and more particularly to a respiratory tube that has a cleanable outside surface.

### BACKGROUND OF THE INVENTION

Respiratory tubes for conveying gases to and/or from a patient have to be light-weight and very flexible to bend easily in order to minimize any forces applied to the patient interface. Minimizing the forces applied to the patient interface enhances patient comfort, which in turn can improve patient compliance. Furthermore, the respiratory tubes have to be resistant to kinking or pinching when bent in order to prevent any impact on or interruption of the gas flow to and/or from the patient. To meet these requirements, state of the art respiratory tubes are typically provided as corrugated—or accordion-type—tubes made of a suitable light-weight plastic material.

The corrugated tubes are constructed having ridges and troughs, which allow the hose to bend and flex. The ridges and troughs form an irregular surface area along the length of the hose.

Unfortunately, the irregular surface area is extremely difficult, if not impossible, to clean and/or disinfect properly, resulting in a buildup of contaminants occurring especially in the troughs of the corrugated tubes. Therefore, corrugated respiratory tubes are typically provided as single-use disposable products, resulting in substantial costs and the creation of substantial amounts of contaminated waste material.

It is desirable to provide a respiratory tube that is light-weight, very flexible to bend easily, and resistant to kinking or pinching while also having an outer surface that is easy to clean.

It is also desirable to provide a respiratory tube that comprises a corrugated tube but has an outer surface that is easy to clean.

### SUMMARY OF THE INVENTION

Accordingly, one object of the present invention is to provide a respiratory tube that is light-weight, very flexible to bend easily, and resistant to kinking or pinching while also having an outer surface that is easy to clean.

Another object of the present invention is to provide a respiratory tube that comprises a corrugated tube but has an outer surface that is easy to clean.

According to one aspect of the present invention, there is provided a cleanable respiratory tube. The cleanable respiratory tube comprises a corrugated tube adapted for conveying gases to and/or from a patient. The corrugated tube has a first and a second fitting at a first and a second end thereof, respectively. A sheath having a smooth cleanable outer surface encases the corrugated tube. A first and a second end of the sheath is fixedly attached to the respective first and second fining. The sheath is made of a flexible foil-type sheet material surrounding the corrugated tube having an inside diameter approximately equal to an outside diameter of ridges of the corrugated tube such that the sheath is in a touching relationship therewith.

According to another aspect of the present invention, them is provided a cleanable respiratory tube. The cleanable respiratory tube comprises a corrugated tube adapted for conveying gases to and/or from a patient. The corrugated tube has a first and a second fitting at a first and a second end thereof, respectively. A sheath having a smooth cleanable outer surface encases the corrugated tube. A first and a second end of the sheath is fixedly attached to the respective first and second fitting. The sheath surrounding the corrugated tube has an inside diameter larger than an outside diameter of ridges of the corrugated tube. The sheath is sufficiently flexible to enable easy bending of the respiratory tube and sufficiently rigid to enable free movement of the corrugated tube within the sheath.

The advantage of the present invention is that it provides a respiratory tube that is light-weight, very flexible to bend easily, and resistant to kinking or pinching while also having an outer surface that is easy to clean.

A further advantage of the present invention is that it provides a respiratory tube that comprises a corrugated tube but has an outer surface that is easy to clean.

### BRIEF DESCRIPTION OF THE DRAWINGS

A preferred embodiment of the present invention is described below with reference to the accompanying drawings, in which:

FIGS. 1a and 1b are simplified block diagrams illustrating in a partial cross sectional view and a side view, respectively, a cleanable respiratory tube according to a preferred embodiment of the invention;

FIG. 1c is a simplified block diagram illustrating in a partial cross sectional view diameters of components of the cleanable respiratory tube according to the preferred embodiment of the invention;

FIG. 1d is a simplified block diagram illustrating in a partial cross sectional view a detail of the cleanable respiratory tube according to the preferred embodiment of the invention;

FIG. 1e is a simplified block diagram illustrating in a side view the outside surface of the cleanable respiratory tube according to the preferred embodiment of the invention;

FIG. 2a is a simplified block diagram illustrating in a partial cross sectional view a cleanable respiratory tube according to another preferred embodiment of the invention;

FIG. 2bis a simplified block diagram illustrating in a partial cross sectional view diameters of components of the cleanable respiratory tube according to the other preferred embodiment of the invention;

FIG. 2c is a simplified block diagram illustrating in a partial cross sectional view a detail of the cleanable respiratory tube according to the other preferred embodiment of the invention;

FIG. 3a is a simplified block diagram illustrating in a side view the cleanable respiratory tube according to the preferred embodiment of the invention employed in a two-leg circuit; and,

FIG. 3b is a simplified block diagram illustrating in a side view the cleanable respiratory tube according to the other preferred embodiment of the invention employed in a two-leg circuit.

### DESCRIPTION OF THE PREFERRED EMBODIMENT

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

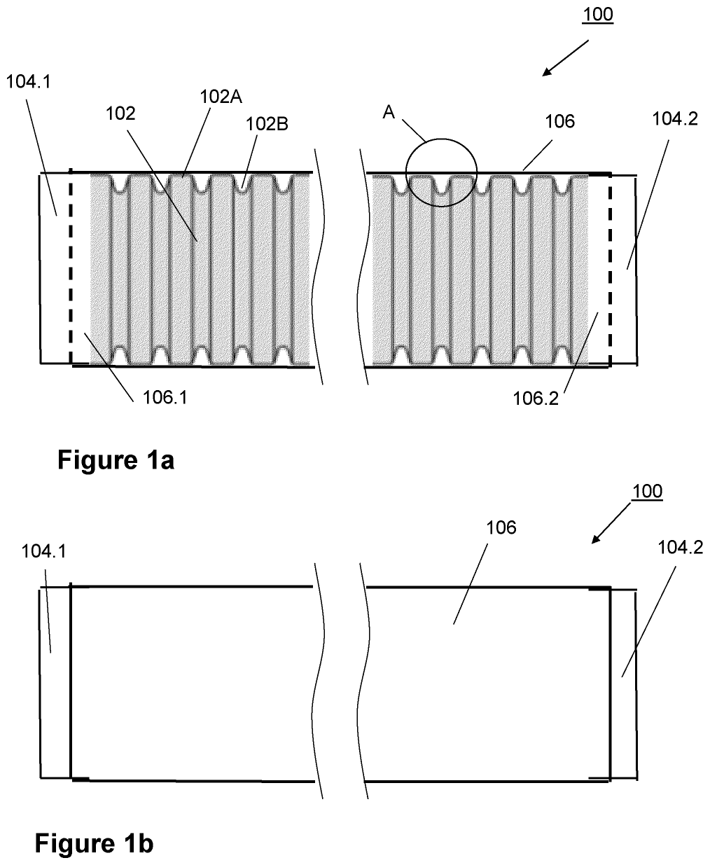

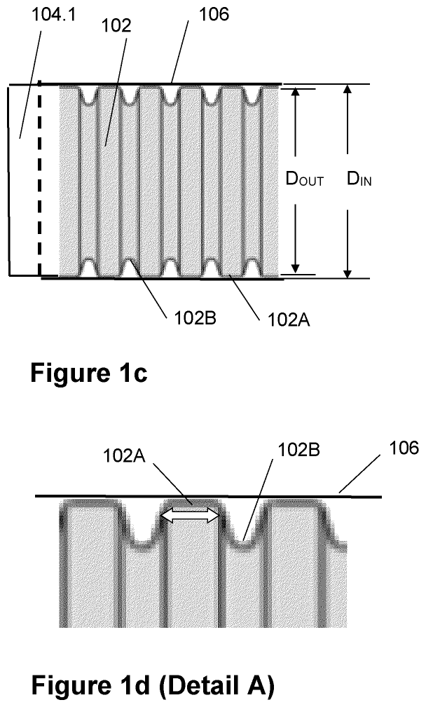

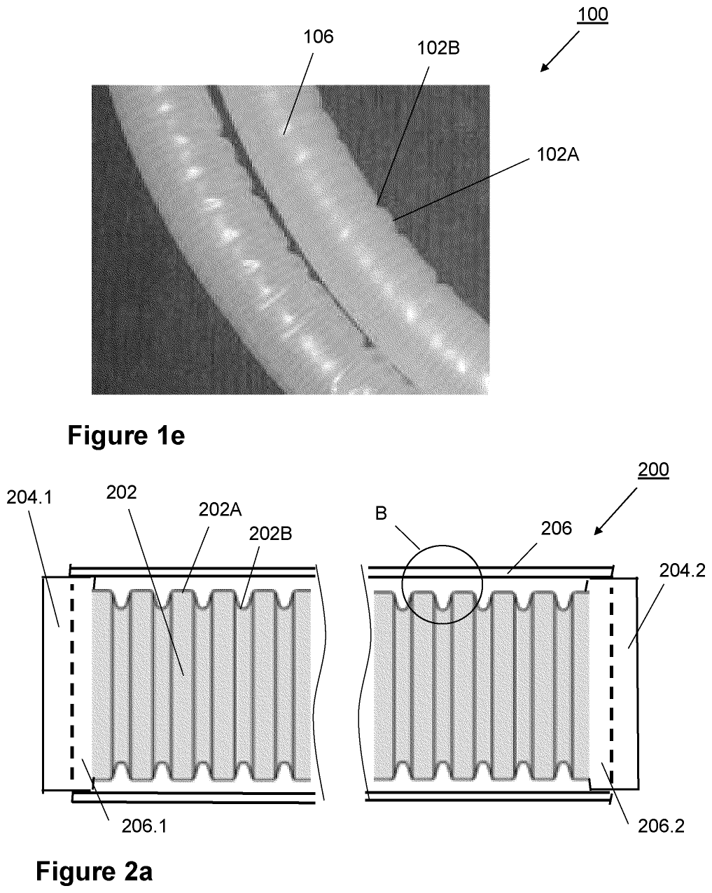

Referring to FIGS. 1a to 1e, a cleanable respiratory tube 100 according to a preferred embodiment of the invention is provided. The cleanable respiratory tube 100 comprises corrugated tube 102 adapted for conveying gases to and/or from a patient. The corrugated tube 102 has a first and a second fitting 104.1 and 104.2, respectively, at a first and a second end thereof. The corrugated tube 102 is, for example, a commercially available corrugated plastic tube for medical applications having sufficient flexibility to bend easily in order to minimize any forces applied to the patient interface during respiration. Typically, the corrugated tube 102 and the fittings 104.1 and 104.2 are provided as a single unit made of the same material.

Sheath 106, having a smooth cleanable outer surface, is encasing the corrugated tube 102. A first and a second end of the sheath 106.1 and 106.2, respectively, are fixedly attached to the respective first and second fitting 104.1 and 104.2. Preferably, the sheath 106 is made of a flexible foil-type sheet material surrounding the corrugated tube 102 and has an inside diameter D_{IN} approximately equal to an outside diameter D_{OUT} of ridges 102A of the corrugated tube 102 such that the sheath 106 is in a touching relationship therewith, as illustrated in FIG. 1c. For example, thin PolyEthylene (PE) sheet material having a thickness of approximately 1 mil is employed for providing sufficient flexibility as well as sufficient strength in order to enable easy bending of the cleanable respiratory tube 100 while preventing rupture of the sheath 106 during bending. The sheet material is applied, for example, by first forming the same into a tube with some overlap being welded using a conventional plastic welding process such as hot air welding and then inserting the corrugated tube 102 therein. After insertion, the first and a second end of the sheath 106.1 and 106.2 are fixedly attached to the respective first and second fitting 104.1 and 104.2 using again a conventional plastic welding process such as hot air welding. Alternatively, the plastic welding process may be replaced using an adhesive. Further alternatively, the sheet material is disposed around the corrugated tube 102 and then welded or glued along the overlap.

The sheath 106 has an inside diameter D_{IN} approximately equal to an outside diameter D_{OUT} of ridges 102A of the corrugated tube 102 such that the same is in a touching relationship with the ridges 102A, i.e. there is substantially no air space between the outside diameter D_{OUT} of ridges 102A and the sheath 106. The only air space is above the troughs 102B between two successive ridges 102A. The sheath 106 being in a touching relationship with the ridges 102A enables some longitudinal movement between the corrugated tube 102 and the sheath 106, as indicated by the block arrow in FIG. 1d. As illustrated in FIG. 1e, the sheath 106 provides a smooth outer surface, which can quickly be wiped down for complete and proper cleaning, while the longitudinal movement between the corrugated tube 102 and the sheath 106 also enables easy bending of the cleanable respiratory tube 100.

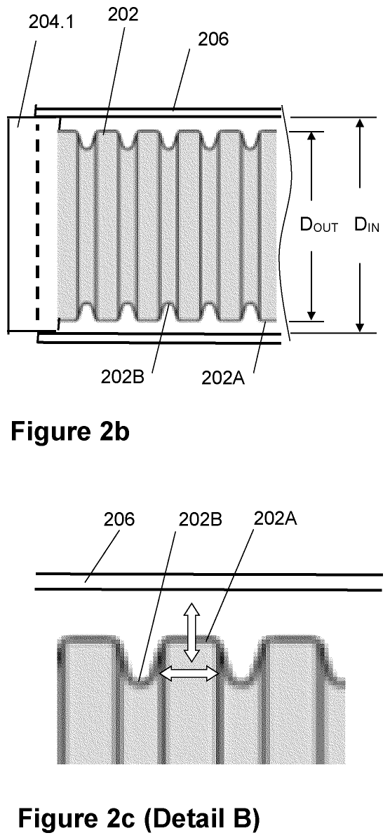

Referring to FIGS. 2a to 2c, a cleanable respiratory tube 200 according to another preferred embodiment of the invention is provided. The cleanable respiratory tube 200 comprises corrugated tube 202 adapted for conveying gases to and/or from a patient. The corrugated tube 202 has a first and a second fitting 204.1 and 204.2, respectively, at a first and a second end thereof. The corrugated tube 202 is, for example, a commercially available corrugated plastic tube for medical applications having sufficient flexibility to bend easily in order to minimize any forces applied to the patient interface during respiration. Typically, the corrugated tube 202 and the fittings 204.1 and 204.2 are provided as a single unit made of the same material.

Sheath 206, having a smooth cleanable outer surface, is encasing the corrugated tube 202. A first and a second end of the sheath 206.1 and 206.2, respectively, are fixedly attached to the respective first and second fitting 204.1 and 204.2. Preferably, the sheath 206 is a flexible smooth wall tube surrounding the corrugated tube 202 and has an inside diameter D_{IN} larger than an outside diameter D_{OUT} of ridges 202A of the corrugated tube 202, as illustrated in FIG. 2b. For example, the smooth wall tube is a commercially available tube made of a suitable plastic material such as PE that is sufficiently flexible to enable easy bending of the cleanable respiratory tube 200 and sufficiently rigid to enable free movement of the corrugated tube 202 within the sheath 206. The corrugated tube 202 is inserted into the sheath 206 and thereafter the first and a second end of the sheath 206.1 and 206.2 are fixedly attached to the respective first and second fitting 204.1 and 204.2 using a conventional plastic welding process such as hot air welding. Alternatively, the plastic welding process may be replaced using an adhesive.

The sheath 206 has an inside diameter D_{IN} larger than an outside diameter D_{OUT} of ridges 202A of the corrugated tube 202 thus providing an air space between the outside diameter D_{OUT} of the ridges 202A and the sheath 206. The sheath 206 having a larger diameter than the ridges 202A enables longitudinal movement between the corrugated tube 202 and the sheath 206 as well as movement perpendicular thereto, as indicated by the block arrows in FIG. 2c. Having the corrugated tube 202 nested inside the smooth wall tube forming sheath 206 provides a smooth outer surface, which can quickly be wiped down for complete and proper cleaning, while provision of the corrugated tube 202 therein prevents kinking.

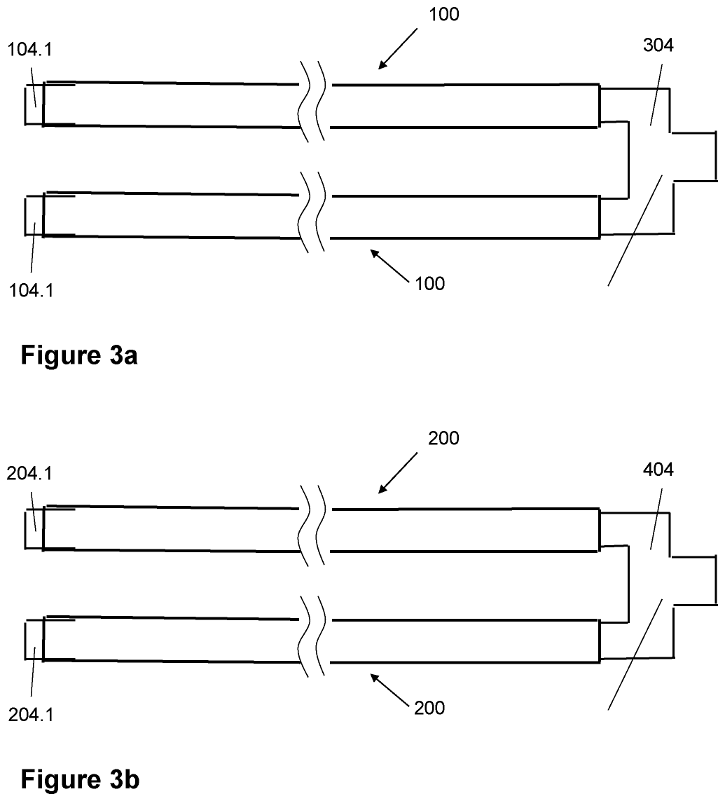

Referring to FIGS. 3a and 3b, the cleanable respiratory tubes 100 and 200, respectively, are employed to form one leg of a multi-leg circuit with respective fittings 304 and 404 being adapted for being connected to two tubes, as illustrated in FIGS. 3a and 3b. As is evident to one skilled in the art, the cleanable respiratory tubes 100 and 200 are also employable to form one leg of a multi-leg circuit with more than two legs.

The present invention has been described herein with regard to preferred embodiments. However, it will be obvious to persons skilled in the art that a number of variations and modifications can be made without departing from the scope of the invention as described herein.

I claim:

1. A cleanable respiratory tube comprising:
a. a corrugated tube adapted for conveying gases to and/or from a patient, the corrugated tube having a first and a second fitting at a first and a second end thereof respectively; and
b. a sheath encasing the corrugated tube, the sheath having a smooth cleanable outer surface, a first and a second end of the sheath being fixedly attached to the respective first and second fitting, wherein the sheath is made of a flexible foil-type sheet material surrounding the corrugated tube having an inside diameter approximately equal to an outside diameter of ridges of the corrugated tube such that the sheath is in a touching relationship therewith.

2. A cleanable respiratory tube comprising:
a. a corrugated tube adapted for conveying gases to and/or from a patient, the corrugated tube having a first and a second fitting at a first and a second end thereof, respectively; and
b. a sheath encasing the corrugated tube, the sheath having a smooth cleanable outer surface, a first and a second end of the sheath being fixedly attached to the respective first and second fitting, wherein the sheath surrounding the corrugated tube has an inside diameter larger than an outside diameter of ridges of the corrugated tube and wherein the sheath is sufficiently flexible to enable easy bending of the respiratory tube and sufficiently rigid to enable free movement of the corrugated tube within the sheath.

3. A multi-leg cleanable respiratory circuit comprising at least two cleanable respiratory tubes according to claim 1 or claim 2.
